(19) United States
(12) Patent Application Publication
(10) Pub. No.: US 2011/0166650 A1
(43) Pub. Date: Jul. 7, 2011

(54) NEW TECHNIQUE FOR PREPARING, STORING AND TRANSPLANTING ENDOTHELIAL GRAFTS

(76) Inventor: Massimo Busin, Forli (IT)

(21) Appl. No.: 13/062,042

(22) PCT Filed: Sep. 4, 2008
(86) PCT No.: PCT/IB2008/053581

§ 371 Date: Mar. 18, 2011

(51) Int. Cl. A61F 2/14; A01N 1/02

(52) U.S. Cl. 623/5.11; 435/1.1; 606/107

(57) ABSTRACT

The present invention relates to a new isolated cornea wherein the Descemet's membrane is separated from the overlying corneal stroma by an air cushion. For “DMEK” surgery a spatula (1) is provided, comprising a rounded glide (2) suitable for receiving on its upper surface (3) two overlapping separated buttons; said overlapping separated buttons comprising a lower button of support and an upper donor button to be introduced into the recipient's eye, a retaining means (4) of said overlapping separated buttons perimetrically disposed on a border (5) of said rounded glide (2) said retaining means (4) defining an aperture (6) suitable for extracting said upper button form said rounded glide (2) and a handle (7) solidly connected to said glide (2) and comprising a first portion (8) and a second portion (9), said first (8) and second (9) portions forming an a angle (10) with respect to each other.

The present invention relates to a new isolated cornea wherein the Descemet's membrane is separated from the overlying corneal stroma by an air cushion, the method for the preparation thereof, a new method for the preparation of a Descemet's membrane-corneal endothelium donor button for use into surgery for transplantation and a new method for transplanting Descemet's membrane and corneal endothelium from a donor in a recipient's eye in need thereof.

### STATE OF THE ART

The cornea represents the anterior portion of the eye shell and is free of blood vessels. Thanks to its transparency and curvature, the cornea behaves as a powerful positive lens, that focuses light on the central part of the retina. The preservation of corneal transparency and physiological curvature is essential for vision.

The cornea consists essentially of three different types of tissue lying adherently on one another, i.e. (from the outside to the inside), epithelium, stroma, and endothelium.

The epithelium consists of several layers of cells kept together very tightly by means of so-called tight-junctions and serves the main purpose of preventing any foreign material or substance or microorganism from penetrating into the deeper layers (protective function).

The stroma builds the skeleton of the cornea, of which represents more than 90% in thickness. It is made of collagen fibrils which run parallel to each other in layers. The direction of the fibres in each layer is orthogonal to the direction of the fibres in the overlying and the underlying layers. In general the distance between the fibrils is lower than half of the wavelength of visible light, and this characteristic results in the transparency of the cornea. If the distance increases, e.g. when water enters the cornea and causes edema, the transparency goes lost.

The more internal layer, is a monolayer of specialised flat cells that lines the posterior surface of the cornea and faces the anterior chamber of the eye, being in contact with the aqueous humour. This monolayer is called endothelium and sits on a basal membrane, which it produces itself, called Descemet membrane. The main function of the endothelium is to keep the hydration of the cornea to a relatively low level (about 70%). This is possible because of the presence of a bicarbonate pump in the endothelial cells, which continuously removes from the cornea the fluid that naturally enters it from the anterior chamber. The mechanism by which said removal is achieved, known as endothelial pump, consists of carriers and ion channels promoting the flux of ions from the stroma to the aqueous humor (mainly Na⁺ and HCO₃⁻) followed by a water movement.

The human corneal endothelium does not show mitotic activity and the number of its cells slowly decreases with age (about 0.5% per year starting from adulthood). Moreover, the loss of corneal endothelial cells can be enhanced by various ocular diseases.

Any endothelial disease resulting in a strong reduction of endothelial cell density also reduces the endothelial pump function. When this reaches a level that is not sufficient to eliminate all the water naturally entering the cornea, the cornea itself becomes thicker (edematous) and looses transparency.

### Corneal Pathologies

All diseases causing a serious alteration of corneal curvature and/or thickness result in partial or complete blindness. If said diseases cannot be healed with therapeutic compositions, the damage they produce is irreversible and corneal transplant represents the sole therapeutic option.

The diseases leading to corneal curvature alterations are represented by keratoconus which is a congenital ectasia (i.e. a condition of inner eye pressure pushing out against a thinned corneal wall, causing it to bulge resulting in worsening vision over time) affecting young individuals and causing a corneal deformation into a more conical shape than its normal gradual curve resulting in a substantial distortion of vision. Transparency alterations comprise congenital diseases, corneal dystrophy, inherited diseases damaging the corneal endothelium or causing an accumulation of anomalous substances in the corneal stroma with consequences on vision that become more severe during adulthood; inflammatory diseases such as the infection produced by Herpes Virus, degenerative diseases such as bullous kerathopathy resulting from failure of the corneal endothelium to maintain the normally dehydrated state of the cornea that can be caused by corneal endothelial trauma and can occur during intraocular surgery (e.g., cataract removal) or after placement of a poorly designed or malpositioned intraocular lens implant, promoting the development of bullous keratopathy; traumatic diseases such as perforations. Finally, a further patient's group is represented by subjects undergoing a further transplantation as a consequence of a rejection or because of the exhaustion of the corneal flap effectiveness. About 50% of corneal transplants is performed because of corneal endothelium disorders.

### Corneal Transplant

Corneal transplant consists in the substitution of the central corneal region with an homologous corneal flap having, normally, a diameter of 8.0-8.5 mm (penetrating keratoplasty, PK). The surgery is performed using corneas explanted from selected donors in total or local anaesthesia and lasts between 30 and 60 minutes. The healthy tissue is sutured at the residual portion of the pathological cornea with a nylon thread 10/0 that is hence left in situ for about 12 to 18 months. The procedure requires few days of hospital-staying or can be performed outpatient surgery; it is followed by a relatively slow visual recovery and usually does not require systemic immunosuppressive therapy. Rejection's risks are extremely limited due to the fact that the cornea does not contain blood vessels. In case of a manifestation of rejection the same can be controlled by topical and systemic steroidal therapy.

In the forties Louis Paufique (1899-1981), a famous French ophthalmologist developed the lamellar keratoplasty (LK) with the aim of substituting only the superficial pathologic layers of an altered cornea hence leaving the deepest, healthy, corneal structures untouched. Depending on the surgical technique, the LK can be either superficial (substitution of the anterior part of corneal stroma), deep (substitution of the whole corneal stroma down to the Descemet's membrane) posterior (substitution of the posterior part of the corneal stroma together with the Descemet's membrane and the corneal endothelium).

In 2002, Anwar and Teichmann developed a deep lamellar technique in order to separate the whole corneal stroma from the underlying Descemet membrane and named it the big bubble technique (Anwar M, Teichmann K D. J Cataract Refract Surg. 2002 March; 28(3):398-403). The procedure, performed on the patient's eye, involves generating a big air bubble between the stroma and the Descemet's membrane. Removal of the stroma exposes the smooth Descemet's membrane. The technique requires the use of a keratoplasty 16 blade ring marker on the cornea, a partial trephination (300 microns) with pre-set depth followed by the insertion of a bent 27 G needle attached to an air filled syringe down into the corneal groove the needle being advanced deep into the paracentral stroma at about 80% depth. Once the needle is correctly in place the plunger is pressed with some force in order to form a bubble recognisable by a white circular circle that allows removal of the stroma anterior to the bubble with a blade. A side entry is created peripherally to the bubble allowing some aqueous to exit from the eye. The bubble is penetrated with a sharp blade (30°) and the knife is withdrawn letting the bubble collapse. A specific spatula is hence inserted into the cavity of the bubble through the opening created by the sharp blade, the stroma above the spatula is sliced with the blade and the residual stroma ins hence removed with specific scissors.

Finally, the Descemet's membrane is stripped off the donor button, the donor stroma and epithelium is sutured into place with 10/0 nylon sutures and the tension adjusted using a keratoscope.

The “big bubble” technique was developed for patients with good endothelium and served the purpose of transplanting all but the endothelium from a donor cornea into e recipient eye.

However, another way to separate Descemet membrane and endothelium from the rest of the cornea by means of an air injection can be used to perform a totally different type of surgery, aimed at replacing only the diseased endothelium and Descemet from the affected cornea with healthy donor endothelium and Descemet. In these patients, the extreme thinness of the tissue to be excised, makes the preparation of the donor graft extremely difficult with the techniques described and employed to date.

### Cornea Preservation

A good preservation of the cornea is crucial for transplants and has the objective of maintaining the tissue characteristics intact. In particular, for use in posterior LK, the cells endothelial cell density and morphology should not be affected during the period between removal from the donor and transplantation into the recipient.

The oldest preservation method was introduced by Filatov in 1935 and consists in the preservation of the whole donor eyeball in a moist chamber. The bulb is wrapped in gauze wetted with sterile saline and stored in a hermetic box. This method is reliable for preservation up to 24 hours. The disadvantages of this method became BETTER known in the sixties when the relevance of corneal endothelium was established in the medical field. The anterior chamber of the eye of a dead individual contains hydrolytic enzymes and toxic metabolites in a concentration that is proportional to the post mortem length. It is hence crucial, in order to preserve the cornea and most of all the endothelium from the detrimental action of said enzymes and metabolites, to isolate it from the ocular bulb as soon as possible and to preserve it in suitable conditions.

In 1974 McCarey and Kaufman developed a liquid solution for preservation of the cornea at 4° C. At that temperature, the metabolism of corneal cells is highly reduced and the endothelial activity is almost absent. Due to stromal hypertonicity the cornea stored in a culture medium swells, causing a delay in the postoperative recovery of corneal transparency. Therefore high-molecular weight dextran (mw 40.000 KDa) was added to the solution to osmotically prevent the influx of fluid into the corneal tissue. Several formulations deriving form the McCarey's solution have been marketed, all comprising a cellular culture medium, one or more antibiotics (i.e. gentamicin and/or others) deturgescent agents (i.e. dextran and chondroitin sulphate), metabolic substrates, antioxidants, growth factors. The corneas preserved at 4° C. undergo degenerative events thus they are preferably transplanted within 7 days (in certain particular cases donor tissues can be kept up to 10-12 days).

In the seventies a new technique for corneal preservation was developed, allowing preservation of corneal tissue for about 4 weeks in culture media supplemented with antibiotics, antimicotics, L-glutammine and growth factors, at 30-37° C. Tissue swelling was eliminated by transferring the isolated cornea (the donor's cornea) into a 4% to 6% solution of high molecular weight dextran (500.000 KDa) about 24 hours prior to use. This 24 hour incubation restores corneal thickness values similar to the original ones. The long preservation period allows the surgeon to plan the surgery, to have ready to use tissues for emergencies, to perform histocompatibility tests and microbiological checks that allow the distribution of microorganisms free tissues.

A further interesting aspect of the tissue culture method of preservation lies in the fact that of the endothelium can undergo a more thorough examination. Furthermore, at the given temperature range, the cornea responds to growth factor stimulation and displays repair activity. Tissues containing initial alterations can hence be recovered for use.

It is evident how tissue preservation is crucial, especially for corneas transplanted in patients with endothelial dysfunction, be it PK or posterior LK.

### Removal of Receipient Endothelium for Posterior LK

Posterior LK is used for substituting endothelium and Descemet, while leaving intact the still healthy anterior corneal layers.

The posterior (endothelial) lamellar keratoplasty (either Deep Lamellar Endothelial Keratoplasty DLEK or Descemet's Stripping Endothelial Keratoplasty DSEK) provides several advantages over the more obsolete PK for the treatment of corneal diseases as only the tissue in need of transplantation is removed and the healthy tissue us hence left intact. Moreover the suturing drawbacks typical of PK are avoided.

In DLEK, a manual deep stromal dissection is carried out to remove a thin posterior stromal lamella together with the underlying endothelium. The procedure is therefore technically challenging with high risk of perforation and most of all the surface obtained with manual dissection is quite rough and of poor optical quality, thus negatively affecting the final visual result.

DSEK is technically easier, because the stromal dissection of the recipient cornea is substituted by simple peeling of Descemet and endothelium from the posterior surface of the recipient cornea, but still requires manual dissection for the preparation of the donor lamella. Waste of donor tissue because of perforation while preparing the graft and the persistence of an interface with a hand-dissected surface are the major disadvantages of this procedure.

A further evolution of DSEK is DSAEK (Descemet Stripping Automated Endothelial Keratoplasty): the only difference consists in the preparation of the donor graft, which in DSAEK is dissected by means of a microkeratome which obtains a perfectly smooth surface also on the donor side of the corneal interface, thus allowing faster visual rehabilitation and better final visual acuity.

Finally, in 2006, Melles published his initial results with the newest technique of endothelial keratoplasty, the so called “Descemet membrane Endothelial Keratoplasty (DMEK)”. In this procedure the donor graft consists purely of Descemet membrane with the underlying endothelium, which is placed on the posterior surface of the recipient cornea after removal of the diseased endothelium and Descemet, thus avoiding the formation of a stromal interface, which may negatively affect vision when present (DSEK and to a lesser extent DSAEK). In addition to the skills required to perform the other types of endothelial keratoplasty, here the surgeon is confronted with the difficult task of removing from the donor cornea the endothelial monolayer together with Descemet membrane, avoiding any possible damage to the tissue to be transplanted With all the techniques described to date the preparation of the donor tissue for DMEK is highly dependent on the surgeon's surgical skills. Preparation of the donor tissue is only one of the challenges the surgeon faces when performing DMEK: handling the “ultra-thin” donor tissue while inserting it into the eye and placing into proper position requires very difficult, painstaking manoeuvres, that to date have prevented this technique from becoming popular in the ophthalmic surgeons community.

### Preparation of the Donor Endothelial/Descemet Graft

To date the only method described to separate donor Descemet's membrane together with the endothelium form the overlying stroma employs manual stripping by means of specially designed micro-instruments (spatula, hook, etc.). As already said, the corneal endothelium is a monolayer of cells lying on its basal membrane (Descemet membrane) and strictly adherent onto the stroma posterior corneal surface; hence it is not surprising that the stripping technique is very difficult and often ineffective, leading to frequent rupture of the donor's tissue of interest. The technique developed by Dr. Melles and is described in US2005/0010244 patent application, requires a tool described in the same application that allows the removal of the Descemet's membrane and the corneal endothelium attached thereto, together with the lower part of the corneal stroma to whom the Descemet's membrane is attached and use of the same for deep LK. In this technique a donor button is incised in the area of the scleral spur and a superficial dissection is performed to separate the posterior corneal layers (thin layer of posterior stroma, the Descemet's membrane and the endothelial layer) from the anterior corneal layers, across the cornea up to the scleral spur over 360 degrees. Another method is described by Z Zuh et al (Cornea. 2006 July; 25(6):705-8) describing a modification of Melles' technique (Cornea 2004:23.286-8) and a sheet of Descemet membrane and endothelium with minimal endothelial damage essentially free from stroma is obtained.

All the above described methods are not the most effective or most handy methods for an easy preparation of the donor endothelial button and require an extremely high skill from the person preparing the same, in fact, when the preparation is made by stripping, it is a healthy endothelium that is stripped form the stroma, the healthy endothelium being well adhering to the corneal stroma as opposed to the scarce adherence and easiness in stripping of the recipient's eye, unhealthy, corneal endothelium. The strong adherence of the cell monolayer makes the stripping of the same a troublesome technique with high risks of mechanically ripping the corneal endothelial layer, which cannot then be used any longer for transplantation. On the other hand, with Melles' technique tools expressly designed for this purpose are necessary and dissection is performed by hand, thus requiring particular skills to manipulate such a thin layer. In particular, as sharp instruments are used, mistakes while performing the dissection will result in improper cuts and tissue damage beyond the possibility of use for transplantation.

Several donor corneas may be therefore discarded due to the difficulty in the preparation of donor endothelium.

### DESCRIPTION

The present invention is related to a new method for the preparation of an isolated eye portion comprising a cornea and its surrounding scleral ring from a donor that can be performed also by a corneal bank technician wherein the Descemet's membrane is spaced from the corneal stroma without employing manual dissection or stripping and wherein the isolated cornea thus prepared can be stored up to about 12 days in corneal storage medium at 30-37° C.

The invention hence, relates to a method for the preparation of an isolated eye portion comprising a cornea and a part of the sclera surrounding said cornea wherein the corneal endothelium together with the Descemet's membrane are spaced from the corneal stroma together with the corneal epithelium by an air cushion and wherein said corneal endothelium together with the Descemet's and said corneal stroma together with the corneal epithelium are sealed by said sclera at the corneal limbus, thus defining said air cushion comprising the steps of:
inserting the needle of a syringe into the scleral margin at about 1-2 mm from the posterior surface of the corneal limbus until reaching the corneal stroma immediately underlying the endothelium of said cornea just beyond the limbus;
injecting air until detachment of the Descemet's membrane form said corneal stroma and further injecting air until obtaining an air cushion between said Descemet's membrane and said corneal stroma, thus conferring to said cornea a spherical shape (bubble formation).

The invention also relates to an isolated eye portion comprising a cornea and a part of the sclera surrounding said cornea, wherein the corneal endothelium together with the Descemet's membrane are spaced from the corneal stroma together with the corneal epithelium by an air cushion and wherein said corneal endothelium together with the Descemet's membrane and said corneal stroma together with the corneal epithelium are sealed at the corneal limbus level by said sclera thus defining said air cushion. The isolated eye portion according to the invention allows an easy and prompt preparation of a button for endothelial keratoplasy (Descemet Membrane Endothelial Keratoplasty—DMEK—) consisting of Descemet's membrane+corneal endothelium.

The invention also relates to a method for the preservation of the isolated eye portion of the invention wherein parts thereof are usable for transplantation comprising the steps of:
culturing said eye portion in a suitable medium at 30-37° C.
preserving said isolated eye portion for a period up to 12 days from its preparation.

The invention relates as well to a method of a preparation of a Descemet's membrane+corneal endothelium button for transplantation and to a surgical method for DMEK using the isolated eye portion comprising a cornea and its surrounding scleral ring of the invention.

The invention further relates to a modified Busin glide suitable for a correct positioning and an easier use of the donor button of the invention in a DMEK surgery.

### DETAILED DESCRIPTION OF THE FIGURES

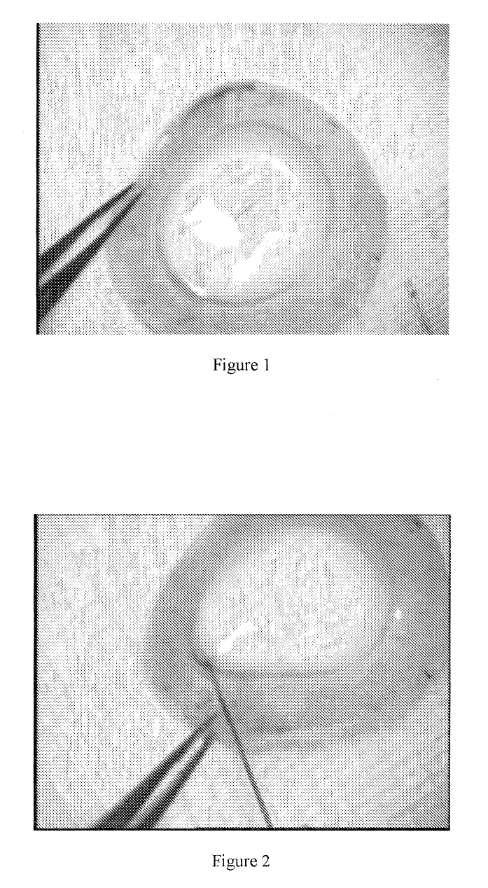

FIG. 1 represents an isolated eye portion comprising a cornea together with its surrounding scleral ring, the scleral ring extending beyond the cornea for about 2-3 mm.

FIG. 2 represents the position of the needle for the preparation of the isolated eye portion comprising a cornea, with the endothelium facing up, together with its surrounding scleral ring of the invention, the image representing the step of positioning the syringe with the needle's opening face up and inserting the thus oriented needle into the corneo-scleral junction (limbus) for about 1-2 mm tangentially to the corneal center, maintaining the needle itself at a stromal level immediately beneath the endothelium.

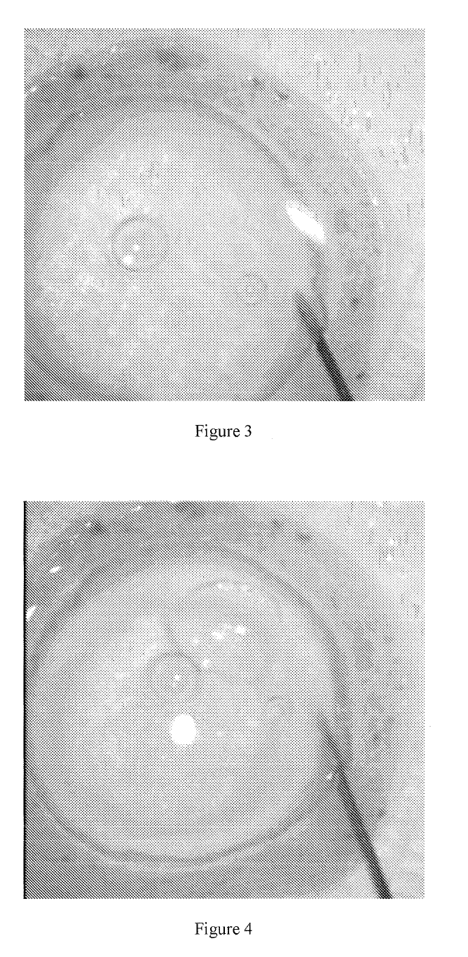

FIG. 3 represents the step of FIG. 2 more in detail, the orientation of the needle and the positioning thereof being clearly visible.

FIG. 4 represents the step of injecting air until visualising the detachment of the Descemet's membrane form said corneal stroma, the beginning of the detachment being visualised in the figure by the formation of a peripheral opaque ring in the corneal region.

FIG. 5 represents a further development of the step represented in FIG. 4, the formation of an air cushion into the eye portion of FIG. 1 being clearly visible.

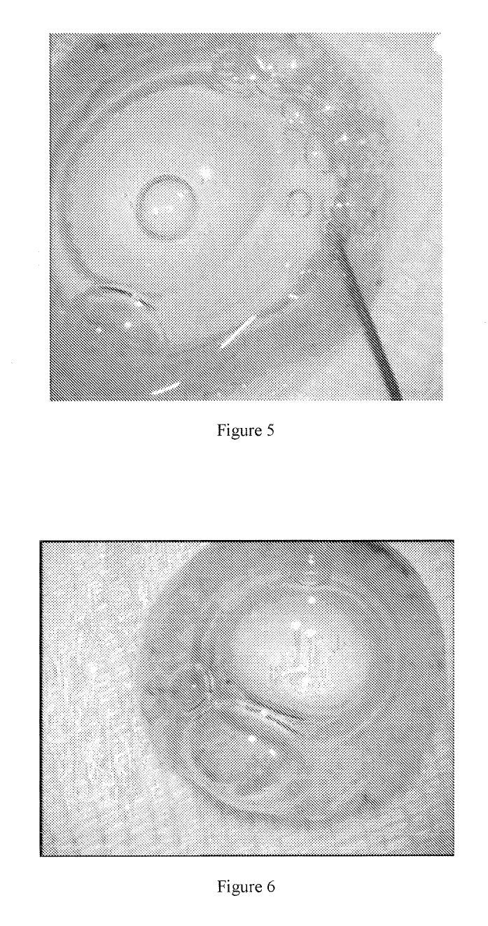

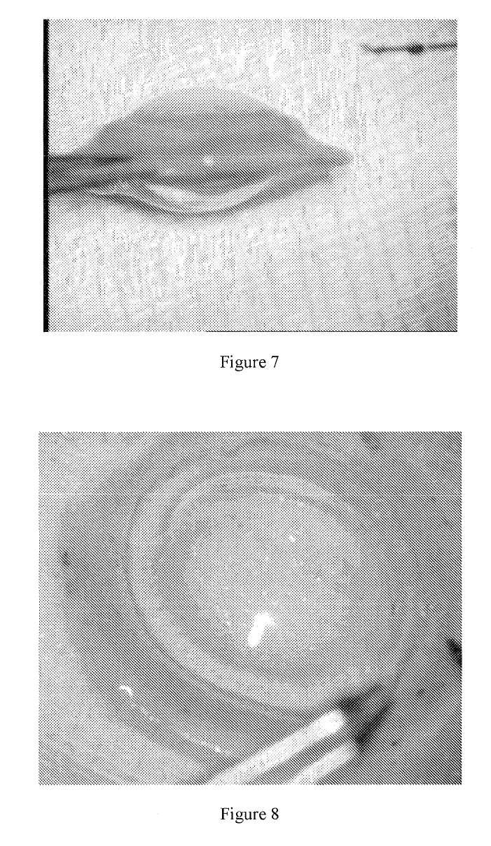

FIGS. 6 and 7 represents the isolated eye portion of the invention comprising a cornea together with its surrounding scleral ring characterised by the presence of an air cushion between the Descemet's membrane and the corneal stroma. The figures show that the air cushion confers to said isolated eye portion a shape resembling the Saturn planet together with its rings, the planet being represented by the inflated cornea and the rings being represented by the flat sclera surrounding the inflated cornea. The smaller bubbles visible are air bubbles in the liquid in which the eye portion was kept or in the liquid used to wash the eye portion.

FIG. 8 shows the position of the needle for the preparation of the donor button consisting of corneal endothelium and Descemet's membrane from the isolated eye portion of the invention.

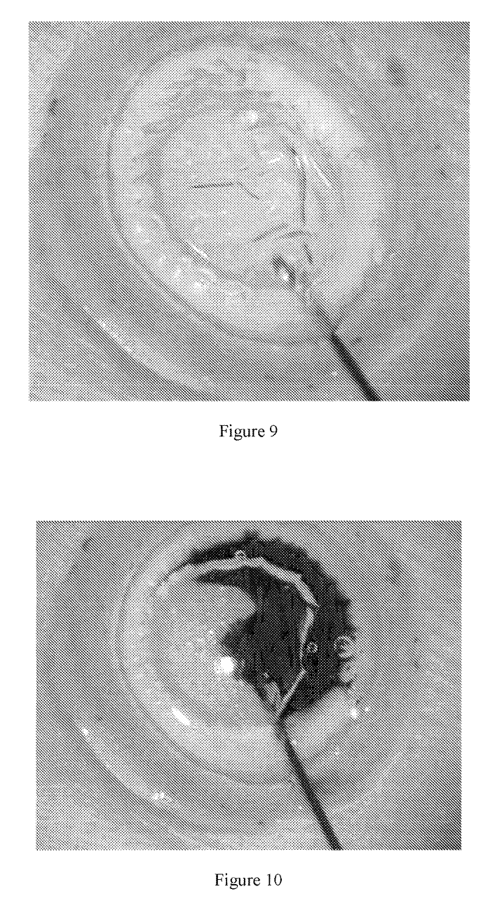

FIG. 9 shows the drawing away of a partial volume of the air of the air cushion separating the corneal stroma and the Descemet's membrane from the isolated eye portion of the invention.

FIG. 10 shows the subsequent filling up of the gap left by the partial air withdrawal with a vital dye (trypan blue) in the isolated eye portion of the invention.

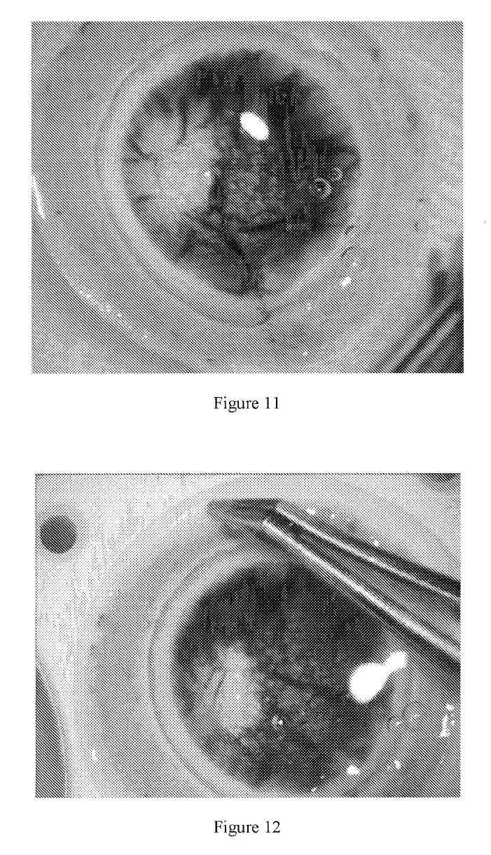

FIG. 11 shows that the remaining air has been drawn from the isolated eye portion of the invention and the layer comprising the Descemet's membrane and the corneal endothelium has collapsed onto the corneal stroma regaining a position similar to the original one and is separated by the corneal stroma by a thin dye layer.

FIG. 12 shows the positioning of the isolated eye portion wherein of FIG. 11 in a punch (the punch being complete with a punch having the desired diameter not shown in figure).

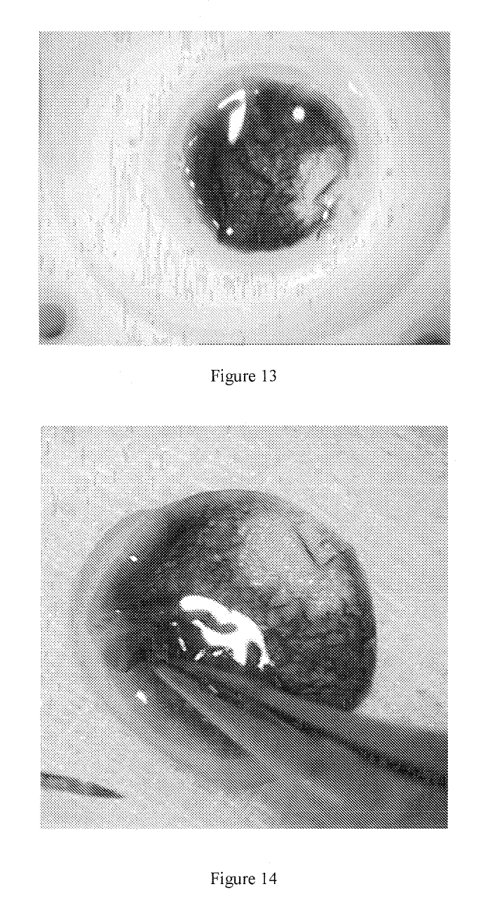

FIG. 13 shows the button resulting from cutting the eye portion of FIG. 12 wherein the corneal stroma button and the button consisting of Descemet's membrane together with the corneal endothelium are separated by a thin layer of dye.

FIG. 14 is a detail of the double button obtained shown in FIG. 2 where it is shown, by pushing on the side with corneal forceps, that the button consisting of the Descemet's membrane together with the corneal endothelium lies flat without rolling up on the corneal stroma.

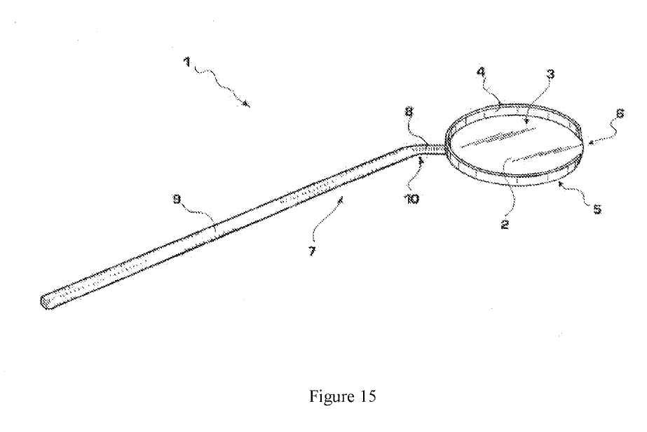

FIG. 15 shows a spatula (1) suitable for carrying out the DMEK of the invention which is a modified Busin glide. The spatula (1) comprises a rounded glide (2), an upper surface (3) a retaining means (4) perimetrically disposed on a border (5) of said rounded glide (2), said retaining means (4) defining an aperture (6) a handle (7) solidly connected to said glide (2) and comprising a first portion (8) and a second portion (9), said first (8) and second (9) portions forming an angle (10) with respect to each other.

### DETAILED DESCRIPTION OF THE INVENTION

Glossary in the Meaning of the Invention:

A definition of terms commonly used in the present specification is given. Unless elsewhere specified, the terms as defined below are as intended in the present specification.

The cornea is the transparent front part of the eye that covers the iris, pupil, and anterior chamber. Together with the lens, the cornea refracts light, and as a result helps the eye to focus, accounting for approximately 80% of the eye's optical power.

Corneal epithelium: a thin epithelial multicellular tissue layer (stratified non-squamous epithelium) of fast-growing and easily-regenerating cells, kept moist by the tear film. Irregularity or edema of the corneal epithelium disrupts the smoothness of the air-tear film interface, the most significant component of the total refractive power of the eye, thereby reducing visual acuity. It is continuous with the conjunctival epithelium and is composed of about 6 layers of cells which are shed constantly on the exposed layer and are regenerated in the basal layer.

Corneal stroma (also substantia propria): a thick, transparent middle layer, consisting of regularly-arranged collagen fibers along with sparsely populated keratocytes. The corneal stroma consists of approximately 200 layers of type I collagen fibrils. 90% of the corneal thickness is composed of stroma.

Descemet's membrane (also posterior limiting membrane): a thin (about 10 μm) acellular layer that serves as the modified basement membrane of the corneal endothelium.

Corneal endothelium: a simple monolayer of mitochondria-rich cells responsible for regulating fluid and solute transport between the aqueous and corneal stromal compartments. The corneal endothelium is bathed by aqueous humour, not by blood or lymph, and has a very different origin, function, and appearance from vascular endothelia.

The corneal limbus is the border of the cornea and the sclera.

The sclera, called the white or white of the eye, is the opaque (usually white, though certain animals, such as horses and lizards, can have black sclera), fibrous, protective, outer shell of the eye containing collagen and elastic fibers. The sclera forms the posterior five sixths of the connective tissue coat of the globe. It is continuous with the dura mater and the cornea, and maintains the shape of the globe, offering resistance to internal and external forces, and provides an attachment for the extraocular muscle insertions.

Corneal punch, is herein intended as a tool comprising a hollow cylindrical blade having a cutting edge allowing to cut a circular section of the donor's cornea.

Donor button as herein intended is a circular section of the cornea of a donor suitable for transplantation, the donor button being intended as consisting of all corneal layers or one or more of said layers. Hence, according to the invention, a donor button may also be a button consisting of the corneal endothelial layer and the Descemet's membrane.

By donor button consisting of corneal endothelium and Descemet's membrane it is herein intended that the donor button is substantially free from other corneal epithelial and stromal tissue. The method for the preparation of the invention might allow some few stromal cells to still adhere to the Descemet's′ membrane, however, no layer (thin or less thin) of corneal stroma is attached to the Descemet's membrane of the button of the invention.

Descemet membrane endothelial keratoplasty (DMEK) is a keratoplasty technique consisting in the transplantation of only the endothelium/Descemet membrane complex.

Clear cornea incision indicates an incision made completely inside the cornea, i.e. beyond the limbus (corneo-scleral junction), through which it is possible to gain access to the structures of the anterior chamber (posterior corneal surface, iris, lens, etc.)

Busin glide is a spatula especially designed for the pull-through technique and allows for an incision as small as 3.2 mm. It facilitates unfolding of the graft, simplifies centration of the donor button in the anterior chamber and minimizes intraoperative manipulation of the graft and the possibility of endothelial loss.

The present description discloses the isolated eye portion as defined above, said eye portion comprising a cornea and a part of the sclera surrounding said cornea wherein the corneal endothelium together with the Descemet's membrane are spaced from the corneal stroma together with the corneal epithelium by an air cushion and wherein said corneal endothelium together with the Descemet's and said corneal stroma together with the corneal epithelium are peripherically sealed by said sclera thus defining said air cushion.

It will be easily understood by the skilled person that an eye portion as the one of the invention, definitely simplifies the surgeon's work in the preparation of the one-cell-layer to be transplanted with DMEK. The eye portion of the invention, that can be preserved in culture up tol2 days, can be provided to the surgeon immediately prior to surgery and will have the advantage of being practically “ready for use”. In fact, the eye portion of the invention provides the surgeon with an intact layer consisting of corneal endothelium and Descemet's membrane already separated from the corneal stroma. It is to be noticed that, although an unhealthy endothelium is easily stripped from the corneal stroma, a healthy endothelium, such as the endothelia needed for the preparation of the donor button in DMEK, is strongly attached to the corneal stroma and a stripping of the same often brings to a rupture of the layer that makes it unsuitable for surgery. Moreover, other means of detachment of the corneal endothelium are time consuming and strongly depend on the surgeons capabilities in manually splitting the cornea for the isolation of the ultra-thin sheet of corneal Descemet and endothelium. It is beyond doubt that the eye portion of the invention strongly advantages the surgeon in the preparation of the donor button.

It is also to be noted that, as the preparation of the eye portion of the invention requires relatively common manual skills (i.e. it does not require special abilities for a surgeon or even for a technician skilled in the field of corneal preparations as it is mainly performed with the aid of air) the separation of the endothelium and Descemet's membrane from the corneal stroma can be performed even by a corneal bank technician. The air injection between the two layers clearly allows to separate the two layers (i.e. Descemet's membrane from corneal stroma) without mechanical insults and risk of ripping the thin endothelial layer.

Hence, the isolated eye portion of the description is suitable for use in the preparation of a button comprising corneal endothelium and Descemet's membrane.

The method for the preparation the isolated eye portion of the description, comprises the following steps:
a needle is inserted into the corneo-scleral junction (limbus) for about 1-2 mm tangentially to the corneal center, maintaining the needle itself at a stromal level immediately under the endothelium. (see as example FIGS. 2 and 3);
air is therein injected until detachment of the Descemet's membrane from the corneal stroma, the detachment of the layers is visible as the formation of a less transparent circle that begins to form in the peripheral region of the cornea and that slowly extends to the central part of the donor cornea (crf FIG. 4 as an example), the injection is hence continued until the creation of an air cushion between said Descemet's membrane and said corneal stroma conferring to said cornea a spherical shape (see figures form 5 to 7 as example).

In the method of the invention, the person preparing the eye portion as herein described, can comfortably proceed in the preparation without particular tools for handling and holding the donor cornea to prepare as described. The isolated eye portion suitable for the preparation of the eye portion of the present description will consist of an explanted cornea surrounded by a small portion of its sclera having the shape of a more or less regular ring. The scleral region normally will extend from the cornea about 2-3 mm, the dimension of the scleral ring being quite irrelevant provided that it is sufficient to seal the peripheral part of the cornea (from about the corneal limbus) thus defining the air cushion spacing the two corneal layers as herein described.

The skilled artisan, hence, can prepare the eye portion of the invention, substantially by positioning an isolated eye portion comprising an intact cornea and a part of the sclera surrounding said cornea on a flat support so that the endothelial part of the cornea lies face up, mounting a needle on a syringe filled with air or on a suitable tool capable of ejecting air; and inserting the needle in proximity of the posterior surface of the corneal limbus until reaching the corneal stroma. It is more handy if the syringe is positioned with the needle's sharp opening face up (as shown in FIG. 3) when inserted in the scleral margin in proximity of the corneal limbus. From the insertion point at the limbus the needle is advanced about 1-2 mm into the clear cornea with a tangential direction maintaining the tip as superficial as possible in the stroma, possibly immediately beneath the endothelium. When proper positioning is achieved, the air in the syringe is injected possibly with a constant pressure and it is possible to visualise the detachment of the Descemet's membrane from the corneal stroma in the form of a forming opaque ring as exemplified in FIG. 4. The air pressure allows the detachment of the two layers without the use of sharp blades, and takes advantage of the adaptability of air to shapes together with the normal elasticity of biological tissues. The detachment by the use of air strongly decreases the chances of ripping of one of the two detached layers. As air is injected until creating a cushion of air reaching substantially the corneal limbus, it is more convenient to use a syringe having a capacity of at least 2.5 cc. While injecting the air, care shall be taken not to over inflate the cavity, in order to avoid rupture of one or more parts. After completing the preparation, the eye portion herein described will have assumed a shape similar to that of the planet Saturn, the central, transparent spherical part comprising the corneal layers and the air cushion and the flat opaque ring around its circumference comprising the sclera as exemplified by FIGS. 6 and 7.

The air injected according to the present description confers to the corneal endothelium plus Descemet's membrane layer an outward convexity which contrast its natural concavity. This inversion of curvature has the practical effect of preventing the endothelium and Descemet's membrane from rolling inwards during all the manoeuvres necessary to first implant and then position it onto the posterior corneal surface. In the state of the art, it is described that the endothelial layer, either stripped or cut away from the stroma, rolls up spontaneously. It is indicated that this rolling up makes the introduction into the anterior chamber quite easy. However, as already mentioned, the donor graft in this type of surgery consists of a single cell layer, and any manipulation necessary to unfold it inside the anterior chamber for correct positioning onto the recipient's posterior corneal surface is extremely difficult and potentially harmful. The rolled up thin layer, in practical terms, has to be manually unfolded inside the anterior chamber of the eye and positioned in order to correctly adhere to the recipient's stroma. The skilled surgeon will appreciate that such procedure is by no means simple or practical.

The inversion of curvature conferred to the isolated eye portion herein described, has the same effect that a rolling up in the opposite direction confers to a rolled up sheet of paper: the sheet of paper flattens.

In a similar way, also the layer consisting of the corneal endothelium and Descemet's membrane of the present description is flattened by creating the air cushion and the button of said layer does not spontaneously roll up once punched from the eye portion of the invention. This flattened button is easily inserted in the recipient's eye by the aid of a glide, such as, by way of example a Busin glide or any suitable glide, and enters the anterior chamber in an unfolded and essentially flat condition thus rendering the positioning of the said button into the recipient's eye anterior chamber extremely simplified.

It has to be kept in mind that the DMEK is a surgical operation carried out on a recipient's eye anterior chamber only through few incisions and is not carried out through a large opening of the said chamber. The correct positioning of the thin donor button, hence, has to be achieved with rather “thin” tools that cannot invade the anterior chamber and that cannot easily flatten up the button. The donor button herein described, hence, has the new feature of being flat as cut and to be more handy for the correct positioning of the same in the recipient's eye anterior chamber. The donor button above described is part of the scope of the invention. Another unpredictable feature of the isolated eye portion of the invention is the fact that it can be preserved in a culture medium such as culture media normally used for corneal preservation at room temperature i.e. 30° C. to 37° C. after preparation. This characteristic could not be predicted as the preservation of the cornea for DMEK, hence a preservation not affecting the corneal endothelium integrity, is up to date achieved only in the very few ways described above. The skilled person could not have predicted whether the isolated eye portion of the invention would have been preservable with known methods for preserving donor's corneas, due to the fact that an air cushion is created in contact with the Descemet's membrane, hence in strict proximity with the endothelium. The presence of air could theoretically damage the endothelium. Surprisingly, if kept deep in a standard corneal preservation medium (e.g. by applying a weight), the eye portion of the invention can be preserved up to 12 days from its preparation without causing substantial changes in the endothelium. Suitable as culture medium are all the usual known cell and tissue culture media for organ cultures of human and animal keratoid integuments. Examples of culture media suitable for corneas are TC 199 (Muller, M. C. et al. in Ophthalmic Res. 20 (1988), pages 44-53), modified TC 199 medium (Reim, M. Klin. Mbl. Augenheilk. 196 (1990), pages 76-80), MEM (minimal essential medium) (Invest Ophthalmol Vis sci 12 (1973), 176-180), modified MEM (cf., for example, Redbrake, C., thesis, Aachen, 1996, page 15; modification of MEM, for example with Earle's salts, with Hank's salts and the like).

The medium will be further enriched with a suitable antibiotic and a suitable antimycotic such as the ones already known in the art for the preservation of cornea. No further explanation should be necessary as the medium for preservation of the corneas in cultures at 30-37° C. are well known in the art as well as the antibiotics and antimicotics to be added to such culture media for a correct preservation of a grafted cornea.

However, donor corneas in which the air has been injected to separate Descemet and endothelium from the stroma, float on the medium and may expose the endothelium to evaporation and mechanical trauma. To prevent this, a small weight (lead or plastic) is tied to the scleral rim, thus sinking the tissue completely into the liquid.

The new isolated eye portion comprising the air cushion herein described requires the carrying out of a new method for the preparation of the donor button comprising corneal endothelium and Descemet's membrane. The present invention hence also relates to a new method for the preparation of a donor button of corneal endothelium and Descemet's membrane suitable for transplantation, from an isolated eye portion of the invention.

The method comprises the following steps starting from comprising a cornea and a part of the sclera surrounding said cornea wherein the corneal endothelium together with the Descemet's membrane are spaced from the corneal stroma together with the corneal epithelium by an air cushion and wherein said corneal endothelium together with the Descemet's and said corneal stroma together with the corneal epithelium are sealed by said sclera at the corneal limbus thus defining said air cushion:
a. removal a partial volume of the air from the air cushion; the volume can be removed, by means of an empty syringe with a needle of about 25, 27, 30 gauge; the removal is carried out so that only a part of the air cushion is removed, therefore the layer consisting of corneal endothelium and Descemet's membrane shrinks slightly and the volume previously occupied by the removed air (hereinafter defined as gap) can be filled up with a small volume of another substance without risking of blowing up the isolated eye portion of the invention;
b. once removed part of the air a vital dye is injected into the gap created by said removing of the air, the injection of dye can be performed with a syringe mounting a needle, such as, by way of example not limiting the invention, a 25, 27 or 30 gauge needle and the dye used is any dye suitable for use in eye surgery that can be introduced into the anterior chamber of the eye without causing permanent colouring or damages of the same such as, by way of example, Trypan blue); once introduced the ink, the remaining air of said air cushion is removed thus causing the layer consisting of the Descemet's membrane and the corneal endothelium to collapse on the corneal stroma, the resulting eye portion comprising a cornea and a part of the sclera surrounding said cornea wherein the corneal endothelium together with the Descemet's membrane are spaced from the corneal stroma together with the corneal epithelium by a layer of ink and wherein said corneal endothelium together with the Descemet's and said corneal stroma together with the corneal epithelium are sealed by said sclera at the peripheral limbus level thus defining said layer of ink;
c. punching the eye portion obtained in step b. thus performing a cut resulting in two overlapping separated buttons, one consisting of corneal stroma and corneal epithelium, another consisting of corneal endothelium and Descemet's membrane wherein said button consisting of corneal endothelium and Descemet's membrane does not roll up spontaneously, a suitable punch can be a punch having a diameter of about 8, 8.5, 9, 9.5, 10 mm. Punches for excision of donor button from a donor's cornea are well known in the art and are available on the market, no further detail is believed necessary on this topic.

As already said, the donor button is characterised in that it consists essentially of corneal endothelium and Descemet's membrane and in that it does not assume spontaneously a rolled up conformation but, instead, is essentially flat.

The present specification also relates to a method of treatment of pathologies by carrying out a transplant of corneal endothelium and Descemet's membrane through a new DMEK.

The new DMEK herein described comprises the steps of
a. preparing or using an already prepared eye portion of the invention;
b. preparing from said eye portion donor button consisting of corneal endothelium and Descemet's membrane as fully described above, the button obtained being essentially flat;
c. removing a button comprising endothelium and Descemet's membrane from the central area of the receiving cornea of the recipient's eye, normally, the button removed in classic DMEK surgeries has a diameter of about 8-10 mm, the same range of sizes is suitable for carrying out the surgical method herein described;
d. preparing 3 clear cornea incisions of about 2 mm each, one in the temporal region of the recipient's cornea, one in the nasal region of the recipient's cornea and one in the superior region of the recipient's cornea,
e. positioning said donor button on a suitable glide with the Descemet's membrane face up and positioning the thus loaded slide close to the nasal clear cut incision of the recipient's eye. Any glide that the skilled surgeon will find suitable, can be used to carry out the present method, such as, by way of a non limiting example, a Busin glide modified in order to accommodate a donor button 8 to 10 mm in diameter and with a 3 mm opening through which the donor tissue can be retracted. A modified Busin glide suitable for the DMEK of the invention is herein described as part of the invention;
f. inserting a coaxial micro incision retinal forceps in the temporal clear cornea incision of the recipient's eye, exiting said coaxial micro incision retinal forceps through the nasal clear cornea incision of the recipient's eye and grabbing with said coaxial micro incision retinal forceps said donor button;
g. dragging inside the recipient's eye anterior chamber said donor button having the Descemet's membrane face up and bring said donor button to adhere to the recipient's corneal stroma by injecting air in said anterior chamber;
h. suturing said clear cornea incisions;
i. injecting a suitable antibiotic and a corticosteroid agent in pharmaceutically effective doses by subconjunctival route.

In the method described above, some steps can be carried out in an order which is not bound to the order indicated in the method's description, in particular, step a. is before step b. that is before step e.; steps c. is before step d.

These two groups of steps shall both be carried out before step f. that is before step g. that is before step h. Nevertheless, a., b. and e. can be indifferently carried out before, after or at the same time of c. and d. As steps a., b. and e. are to be carried out on the donor tissue and steps c. and d. on the recipient's eye, they can also be carried out simultaneously by two different persons. Step a. envisages the possibility of either preparing from a classic donor cornea the isolated eye portion comprising the air cushion of the invention as previously described in detail in the present specification, or using an already prepared eye portion comprising the air cushion of the invention either directly provided as such by a corneal bank either obtained as a classical donor craft from the bank and prepared as described herein even 12 days before the surgery as a “ready to use” product.

The air remaining in the recipient's anterior chamber at point g is left in the chamber to reabsorb spontaneously in few days.

Suitable antibiotics at step i. are: gentamicin or tobramicin and suitable corticosteroids t are, by way of example dexamethasone, the said compounds being already used in eye surgery, the effective dosage being well known in the art.

As already described, a Busin glide is a spatula especially designed for the pull-through technique through an incision as small as 3.2 mm. It facilitates unfolding of the graft, simplifies centration of the donor button in the anterior chamber and minimizes intraoperative manipulation of the graft and the possibility of endothelial loss. This glide, although particularly useful for surgeries known in the art, is not the best tool for carrying out the DMEK of the invention.

The DMEK method of the invention is based on the new, flat membrane-like donor button obtained by the isolated eye portion of the invention as described.

As already indicated, after the punch of the eye portion of the invention, a double button is obtained, consisting of two layers, one layer represented by donor corneal epithelium and donor corneal stroma, the other layer represented by a flat donor corneal Descemet's membrane and donor corneal endothelium.

The flatness of the endothelial button, renders glides with a funnel shaped portion as the Busin glide less suitable as the passage through the funnel shaped region can damage the very thin endothelium plus Descemet button of the invention.

A new glide suitable for the button of the invention is herein disclosed. It has to be noted that the “superposed double button” as obtained with the method herein disclosed has a practical advantage for the surgeon as the thick epithelial and stromal button on which the thin endothelium and Descemet button lays, functions as a support for the thin button keeping the button safe form mechanical damages when handled.

The new glide of the invention is hence designed in order to carry both buttons (the thicker button laying directly on the glide with the thinner button on top) and has a lateral barrier with a small opening designed so to hold the thicker button while allowing the passage of the microincision retinal forceps through the opening for grabbing and pulling inside the recipient's anterior chamber the thin donor button directly through the clear corneal incision.

The modified Busin glide of the invention is represented in FIG. 15.

More in detail the modified Busin glide of the invention is a spatula (1) for a “DMEK” surgery, comprising:
1. a rounded glide (2), suitable for receiving on its upper surface (3) two overlapping separated buttons; said overlapping separated buttons comprising a lower button of support and an upper donor button to be introduced into the recipient's eye;
2. a retaining means (4) of said overlapping separated buttons perimetrically disposed on a border (5) of said rounded glide (2), said retaining means (4) defining an aperture (6) suitable for extracting said upper button form said rounded glide (2); and
3. a handle (7) solidly connected to said glide (2) and comprising a first portion (8) and a second portion (9), said first (8) and second (9) portions forming an angle (10) with respect to each other.
   As explained above, the endothelial and Descemet donor button is maintained on top of the epithelial and stromal button obtained with the punching of the method of the invention that serves as support. Any suitable support for the thin epithelial and Descemet donor button could be suitable.

The spatula of the invention can be made either of a sterile disposable material like plastic suitable for surgery known to the skilled person, or of any other material which is sterilisable and suitable for surgery as normally used alloys known to the skilled person, for a non disposable tool.

The handle of the spatula comprises an angle (10) as indicated in FIG. 15, that has the advantage of being easily positioned by the surgeon on the nasal side of the cornea (the donor's button enters in the anterior chamber from the nasal clear cornea incision) without slipping out of the glide when positioned on the nasal bone, the angle is comprised between 90° and 170°.

The wideness of the aperture (6) from which the donor button is grabbed is substantially comprised between 2 mm and 4 mm and is preferably about 3 mm.

The glide (2) has a diameter of about 8-12 mm. The border height is about 1-2 mm.

The new DMEK herein described can be carried on any patient in need thereof, by way of example, said patient could be affected by any type of endothelial dysfunction leading to corneal edema and loss of transparency with substantial loss of visual acuity. As previously mentioned, the endothelial disease can be primary, as in several corneal dystrophies (Fuchs corneal dystrophy, cornea guttata, congenital hereditary endothelial dystrophy, posterior polymorphous dystrophy), or secondary to surgical trauma occurred during any type of intraocular surgery, but most often during cataract surgery with intraocular lens implantation. Finally, a third indication is posed by corneal grafts previously performed and that have failed either because of immunologic rejection or because of slow endothelial decay. Presently these three indications amount to about half of the total indications for corneal transplantation.

1. An isolated eye portion comprising a cornea and a part of the sclera surrounding said cornea wherein the corneal endothelium together with the Descemet's membrane are spaced from the corneal stroma together with the corneal epithelium by an air cushion and wherein said corneal endothelium together with the Descemet's membrane and said corneal stroma together with the corneal epithelium are sealed at the corneal limbus level by said sclera thus defining said air cushion.

2. The isolated eye portion of claim 1 for use in the preparation of a button comprising corneal endothelium and Descemet's membrane.

3. Method for the preparation of an isolated eye portion comprising a cornea and a part of the sclera surrounding said cornea wherein the corneal endothelium together with the Descemet's membrane are spaced from the corneal stroma together with the corneal epithelium by an air cushion and wherein said corneal endothelium together with the Descemet's and said corneal stroma together with the corneal epithelium are sealed by said sclera at the corneal limbus thus defining said air cushion comprising the steps of:
inserting a needle of a syringe into the posterior surface of a corneal donor tissue through the corneal limbus and advancing the needle until reaching the corneal stroma immediately beneath the endothelium of said cornea;
injecting air until detachment of the Descemet's membrane form said corneal stroma and further injecting air until creating an air cushion between said Descemet's membrane and said corneal stroma conferring to said cornea a spherical shape.

4. The method of claim 3 wherein said needle is inserted into the tissue at the limbus and advanced about 1-2 mm tangentially from said point of insertion at the posterior surface of the corneal limbus.

5. The method of claim 3, wherein said syringe is of at least 2.5 cc.

6. The method of claim 3, wherein said air cushion confers to the Descemet's membrane and corneal epithelium a curvature opposite to the naturally occurring one.

7. The method of claim 3, wherein air is injected with a needle selected between needles of 25, 27, or 30 gauge.

8. Method for the preservation of the isolated eye portion of claim 1 wherein parts thereof are usable for transplantation comprising the steps of:
culturing said eye portion in a suitable culture medium comprising a suitable antibiotic and a suitable antimycotic at 30-37° C.
preserving said isolated eye portion for a period of up to 12 days from its preparation.

9. Method for the preparation of a donor button of corneal endothelium and Descemet's membrane suitable for transplantation, from an isolated eye portion comprising a cornea and a part of the sclera surrounding said cornea wherein the corneal endothelium together with the Descemet's membrane are spaced from the corneal stroma together with the corneal epithelium by an air cushion and wherein said corneal endothelium together with the Descemet's and said corneal stroma together with the corneal epithelium are sealed by said sclera at the corneal limbus level thus defining said air cushion comprising the steps of:
a. removing a partial volume of the air from said air cushion;
b. injecting a suitable vital dye in the gap created by said removing of the air and subsequently removing the remaining air of said air cushion thus causing the layer consisting of the Descemet's membrane and the corneal endothelium to collapse on the corneal stroma, the resulting eye portion comprising a cornea and a part of the sclera surrounding said cornea wherein the corneal endothelium together with the Descemet's membrane are spaced from the corneal stroma together with the corneal epithelium by a layer of dye and wherein said corneal endothelium together with the Descemet's and said corneal stroma together with the corneal epithelium are sealed by said sclera at the peripheral limbus level thus defining said layer of dye;
c. punching the eye portion obtained in step b. thus performing a cut resulting in two overlapping separated buttons, one consisting of corneal stroma and corneal epithelium, another consisting of corneal endothelium and Descemet's membrane wherein said button consisting of corneal epithelium and Descemet's membrane does not roll up spontaneously.

10. The method of claim 9 wherein the punching is performed with a punch having a diameter of about 8, 8.5, 9, 9.5, 10 mm.

11. The method of claim 9, wherein air is injected or removed and ink is injected with a needle selected between needles of 25, 27, or 30 gauge.

12. The method of claim 9, wherein said dye is Trypan Blue or indocyanine green.

13. Donor button consisting of corneal endothelium and Descemet's membrane obtainable with the method of claim 9.

14. Method of treatment of pathologies requiring a transplant of the corneal endothelium comprising the steps of
a. preparing or using an already prepared eye portion comprising a cornea and a part of the sclera surrounding said cornea wherein the corneal endothelium together with the Descemet's membrane are spaced from the corneal stroma together with the corneal epithelium by an air cushion and wherein said corneal endothelium together with the Descemet's and said corneal stroma together with the corneal epithelium are peripherically sealed by said sclera thus defining said air cushion;
b. preparing from said eye portion donor button consisting of corneal endothelium and Descemet's membrane;
c. removing a button comprising endothelium and Descemet's membrane from the central area of the receiving cornea of the recipient's eye;
d. preparing 3 clear cornea incisions of about 2 mm each, one in the temporal region of the recipient's cornea, one in the nasal region of the recipient's cornea and one in the superior region of the recipient's cornea;
e. positioning said donor button on a suitable glide with the Descemet's membrane face up and positioning the thus loaded slide close to the nasal clear cut incision of the recipient's eye;
f. inserting a coaxial microincision retinal forceps in the temporal clear cornea incision of the recipient's eye, exiting said coaxial microincision retinal forcep from the nasal clear cornea incision of the recipient's eye and grabbing with said coaxial microincision retinal forcep said donor button;
g. dragging inside the recipient's eye anterior chamber said donor button having the Descemet's membrane face up and bring said donor button to adhere to the recipient's corneal stroma by injecting air in said anterior chamber;
h. suturing said clear cornea incisions;
i. injecting a suitable antibiotic and a suitable anti-inflammatory immunodepressant agent in pharmaceutically effective doses by subconjunctival route.

15. The method of claim 14 wherein the button comprising endothelium and Descemet's membrane from the central area of the receiving cornea of the recipient's eye has a diameter of about 8-10 mm.

16. The method of claim 14, wherein the glide is a Busin glide or a modified Busin glide.

17. The method of claim 14, wherein the pathology is any type of endothelial dysfunction leading to corneal edema and loss of transparency with substantial loss of visual acuity.

18. The method of claim 17 wherein the endotehelial disease is selected between:
primary diseases comprising corneal dystrophies, Fuchs cornal dystrophy, cornea guttata, congenital hereditary endothelial dystrophy, posterior polymorphous dystrophy;
secondary diseases comprising diseases due to surgical trauma occurred during any type of intraocular surgery, during cataract surgery with intraocular lens implantation;
diseases due to rejection or slow endothelial decay of grafts previously performed and that have failed either because of immunologic rejection or because of slow endothelial decay.

19. A spatula (1) for a “DMEK” surgery, comprising:
a rounded glide (2), suitable for receiving on its upper surface (3) two overlapping separated buttons; said overlapping separated buttons comprising a lower button of support and an upper donor button to be introduced into the recipient's eye;
a retaining means (4) of said overlapping separated buttons perimetrically disposed on a border (5) of said rounded glide (2), said retaining means (4) defining an aperture (6) suitable for extracting said upper button form said rounded glide (2); and
a handle (7) solidly connected to said glide (2) and comprising a first portion (8) and a second portion (9), said first (8) and second (9) portions forming an angle (10) with respect to each other.

20. The spatula (1) according to claim 19, made of a disposable material.

21. The spatula (1) according to claim 19, made of sterilizable material suitable for surgery.

22. The spatula (1) according to claim 19, wherein said angle (10) is comprised between 90° and 170°.

23. The spatula (1) according to, claim 19, wherein said aperture (6) has a wideness along said border (5) is of about 2, 3 or 4 mm, said glide (2) has a diameter of about 8-12 mm, and said border has a height of about 1 to 2 mm.
